(19) United States
(12) Patent Application Publication
(10) Pub. No.: US 2023/0272062 A1
(43) Pub. Date: Aug. 31, 2023

(54) Treatment Of Lung Disease Based Upon Stratification Of Polygenic Score Relating To Response To A Therapeutic Agent

(71) Applicant: Regeneron Pharmaceuticals, Inc., Tarrytown, NY (US)

(72) Inventors: Charles Paulding, Tarrytown, NY (US); Shan Chen, Tarrytown, NY (US)

(21) Appl. No.: 18/059,719

(22) Filed: Nov. 29, 2022

Related U.S. Application Data
(60) Provisional application No. 63/284,041, filed on Nov. 30, 2021.

(51) Int. Cl. C07K 16/24

(52) U.S. Cl. CPC C07K 16/244; A61K 2039/505

(57) ABSTRACT

The present disclosure provides methods of identifying subjects having a lung disease or at risk of developing a lung disease that will respond favorably to treatment with a therapeutic agent that treats or inhibits a lung disease, such as dupilumab, an inhaled corticosteroid (ICS), or a long-acting beta agonist (LABA), and methods of treating a subject having a lung disease with a therapeutic agent that treats or inhibits a lung disease, such as dupilumab, an ICS, or a LABA, and determining or having determined the subject's FEV1 polygenic score (FEV1-PS).

### REFERENCE TO SEQUENCE LISTING

This application includes a Sequence Listing filed electronically as an XML file named 381203561SEQ, created on Nov. 28, 2022, with a size of 16 kilobytes. The Sequence Listing is incorporated herein by reference.

### FIELD

The present disclosure relates to the field of therapeutic treatments of lung diseases. More specifically, the disclosure relates to the methods of identifying subjects having a lung disease that will respond to a therapeutic agent that treats or inhibits the lung disease.

### BACKGROUND

Asthma is an inflammatory disease of the airways of the lungs. The condition is characterized by variable and recurring symptoms, reversible airflow obstruction, and bronchospasms that are easily triggered. The diagnosis of asthma can involve spirometry lung function testing, including determination of the forced expiratory volume in one second (FEV1), and the peak expiratory flow rate, as well as assessment of the annualized exacerbation rate. Treatments of asthma include administration of medications that are fast acting or effective in the longer term. Salbutamol and albuterol are the mainstays of fast-acting medications, whereas inhaled corticosteroids (ICSs) were for many years the cornerstone of long-term therapies. More recently, antibodies such as mepolizumab, dupilumab, and omalizumab have been used in connection with specific types of asthma. However, it is difficult to predict whether a given subject will respond to a given antibody therapy. A genome-wide association study (GWAS) in connection with asthma has been carried out (Moffat et al., N. Engl. J. Med., 2010, 363, 1211-1221). However, Moffat does not disclose methods for identifying subjects having lung disease that will respond to a therapeutic agent that treats or inhibits a lung disease.

Chronic obstructive pulmonary disease (COPD) is characterized in part by air-flow restriction, emphysema and chronic bronchitis. COPD frequently worsens with everyday activity making routine tasks difficult. While tobacco smoke is a major risk factor, other factors include pollution, genetics, and exposure to workplace dusts and chemicals. Diagnosis of COPD frequently comprises spirometry including determination of a patient's FEV1 value. Current treatments include smoking cessation, short-acting bronchodilators, phosphodiesterase-4 inhibitors, corticosteroids, and, in serious cases, antibiotics. However, it is difficult to predict whether a patient will respond to a given treatment.

### SUMMARY

The present disclosure provides methods of treating a subject having a lung disease or at risk of developing a lung disease with a therapeutic agent that treats or inhibits a lung disease, the method comprising: determining or having determined the subject's FEV1 polygenic score (FEV1-PS), wherein the FEV1-PS comprises an aggregate of a plurality of genetic variants associated with pre-bronchodilator FEV1; and administering or continuing to administer the therapeutic agent that treats or inhibits a lung disease at a standard dosage amount when the subject's FEV1-PS is greater than or equal to a threshold FEV1-PS; or administering the therapeutic agent that treats or inhibits a lung disease at dose that is greater than a standard dosage amount when the subject's FEV1-PS is less than the threshold FEV1-PS.

The present disclosure also provides methods of determining whether a subject having a lung disease or at risk of developing a lung disease will adequately respond to treatment with a therapeutic agent that treats or inhibits a lung disease, the method comprising: determining or having determined the subject's FEV1 polygenic score (FEV1-PS), wherein the FEV1-PS comprises an aggregate of a plurality of genetic variants associated with pre-bronchodilator FEV1; wherein: an FEV1-PS that is greater than or equal to a threshold FEV1-PS indicates the subject will respond adequately to treatment with the therapeutic agent that treats or inhibits a lung disease at a standard dosage amount; or an FEV1-PS that is less than a threshold FEV1-PS indicates the subject will not respond adequately to treatment with a standard dosage amount of the therapeutic agent that treats or inhibits a lung disease.

### BRIEF DESCRIPTION OF THE DRAWINGS

The accompanying figures, which are incorporated in and constitute a part of this specification, illustrate several features of the present disclosure.

FIG. 1 shows that the FEV1-PS was significantly associated with an increased change of FEV1 at week 12 in both dupilumab-treated and placebo-treated European subjects (Panel A); and dupilumab-treated patients in the highest quartile showed the greatest improvement in FEV1, compared to placebo-treated patients (Panel B).

FIG. 2 shows that the FEV1-PS was significantly associated with a longitudinal increase of change of FEV1 in both dupilumab-treated and placebo-treated European subjects; in both dupilumab-treated and placebo-treated patients, the patients with the highest FEV1-PS had the greatest FEV1 improvement.

FIG. 3 (Panel A and Panel B) shows that the FEV1-PS was not associated with differences in annualized exacerbation rates in both dupilumab-treated and placebo-treated European subjects.

FIG. 4 (Panel A and Panel B) shows that the FEV1-PS determined from European samples in the U.K. Biobank was significantly associated with pre-bronchodilator FEV1 in GHS_GSA European samples.

FIG. 5 shows a genome-wide FEV1-PS in GHS_GSA European subjects showing an association to exacerbations in asthmatic patients.

FIG. 6 shows a genome-wide FEV1-PS in GHS_GSA European subjects versus asthma (Panel A) and COPD (Panel B).

FIG. 7 shows a genome-wide FEV1-PS (source dataset: GWAS from FEV1 GWAS in subjects without asthma or COPD) associated with exacerbations in asthmatic patients.

FIG. 8 shows a genome-wide FEV1-PS (source dataset: GWAS from FEV1 GWAS in subjects without asthma or COPD) in GHS_GSA European patients shows an association of FEV1-PS to asthma (Panel A) and COPD (Panel B).

### DESCRIPTION OF EMBODIMENTS

Genetic factors can play an important role in the risk of developing diseases, and potentially influence how individuals respond to drug treatment. Polygenic risk scores (PRSs) combine information from a large number of genetic variants, derived from disease association studies, to create a single composite quantitative measure for each individual which reflects the individual's genetically-derived disease risk. An individual with a larger number of risk alleles for a particular disease will have a higher PRS than an individual with fewer alleles for the same particular disease. Risk can be evaluated at several thresholds, such as percentiles, standard deviation units of the population distribution, or absolute values. The present disclosure relates generally to the unexpected finding that stratification of subjects by FEV1-PS is useful in the identification of subjects likely to respond to a therapeutic agent that treats or inhibits a lung disease, including, without limitation, a bronchodilator, an IL-33 antagonist, an interleukin-4 receptor alpha antagonist, and/or an interleukin-13 receptor antagonist.

Spirometry is a common breathing test that measures the maximum force of a patient's breath after inhalation and exhalation. Patients with asthma or other breathing problems may perform spirometry pre- and post-bronchodilator in order to examine the effectiveness of the medication on their breathing problems.

FEV1 is measured by spirometry and it is defined as Forced Expiratory Volume in the first second. The volume of air that can be forced out in one second after taking a deep breath is an important measure of pulmonary function.

FEV1-PS is an aggregate of a plurality of genetic variants associated with the pulmonary metric FEV1. FEV1-PS are calculated, for example, in a representative hypothetical GWAS in Table 2. A GWAS may have identified 4 genetic variants associated with FEV1. Each of the genetic variants may be associated with one or more genes. A value, such as an Odds Ratio, can be calculated for each individual genetic variant. A particular subject's FEV1-PS can be determined by multiplying the log value of the individual Odds Ratio for each variant by the Number Effect Alleles (which is the number of copies of the genetic variant in the genome; i.e., either 0, 1, or 2), and then summing the resultant values. See Table 2 for illustration purpose.

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates (such as, for example, apes and monkeys). In some embodiments, the subject is a human. In some embodiments, the subject is a patient under the care of a physician.

The present disclosure provides methods of treating a subject having a lung disease with a bronchodilator, an IL-33 antagonist, an interleukin-4 receptor alpha antagonist, and/or an interleukin-13 receptor antagonist. In some embodiments, the subject having a lung disease is treated with dupilumab. The methods comprise determining or having determined the subject's therapeutic polygenic score (FEV1-PS). The FEV1-PS comprises an aggregate of a plurality of genetic variants associated with pre-bronchodilator FEV1. The methods comprise administering or continuing to administer dupilumab (or a bronchodilator, an IL-33 antagonist, an interleukin-4 receptor alpha antagonist, and/or an interleukin-13 receptor antagonist generally) at a standard dosage amount when the subject's FEV1-PS is greater than or equal to a threshold FEV1-PS. The methods also comprise administering dupilumab (or a bronchodilator, an IL-33 antagonist, an interleukin-4 receptor alpha antagonist, and/or an interleukin-13 receptor antagonist generally) at a dosage amount that is greater than a standard dosage amount when the subject's FEV1-PS is less than the threshold FEV1-PS. In some embodiments, the standard dosage amount of dupilumab is 200 mg. In some embodiments, the amount of dupilumab that is greater than a standard dosage amount is greater than 200 mg.

In some embodiments, the methods further comprise administering an interleukin-4 receptor alpha antagonist and/or an interleukin-13 receptor antagonist other than dupilumab to a subject having a FEV1-PS that is less than the threshold FEV1-PS. In some embodiments, the methods further comprise administering an IL-33 antagonist to a subject having a FEV1-PS that is less than the threshold FEV1-PS.

The present disclosure also provides methods of treating a subject having a lung disease with a bronchodilator, an IL-33 antagonist, an interleukin-4 receptor alpha antagonist, and/or an interleukin-13 receptor antagonist. In some embodiments, the subject having a lung disease is treated with dupilumab. In some embodiments the subject is administered or continued to be administered dupilumab (or a bronchodilator, an IL-33 antagonist, an interleukin-4 receptor alpha antagonist, and/or an interleukin-13 receptor antagonist generally) at a standard dosage amount, wherein the subject has a FEV1-PS that is greater than or equal to a threshold FEV1-PS. In some embodiments, the subject's FEV1-PS has been previously determined. In some embodiments the subject is administered or continued to be administered dupilumab (or a bronchodilator, an IL-33 antagonist, an interleukin-4 receptor alpha antagonist, and/or an interleukin-13 receptor antagonist generally) at a dosage amount that is greater than a standard dosage amount, wherein the subject has a FEV1-PS that is less than the threshold FEV1-PS. In some embodiments, the subject's FEV1-PS has been previously determined. In some embodiments, the standard dosage amount of dupilumab is 200 mg. In some embodiments, the amount of dupilumab that is greater than a standard dosage amount is greater than 200 mg. In some embodiments, the methods further comprise administering an interleukin-4 receptor alpha antagonist and/or an interleukin-13 receptor antagonist other than dupilumab to a subject having a FEV1-PS that is less than the threshold FEV1-PS. In some embodiments, the subject's FEV1-PS has been previously determined. In some embodiments, the methods further comprise administering an IL-33 antagonist to a subject having a FEV1-PS that is less than the threshold FEV1-PS. In some embodiments, the subject's FEV1-PS has been previously determined.

The present disclosure also provides methods of determining whether a subject having a lung disease or at risk of developing a lung disease will adequately respond to treatment with dupilumab (or a bronchodilator, an IL-33 antagonist, an interleukin-4 receptor alpha antagonist, and/or an interleukin-13 receptor antagonist generally). The methods comprise determining or having determined the subject's FEV1-PS. The FEV1-PS comprises an aggregate of a plurality of genetic variants associated with pre-bronchodilator FEV1. A FEV1-PS that is greater than or equal to a threshold FEV1-PS indicates the subject will respond adequately to treatment with a standard dosage amount of dupilumab (or a bronchodilator, an IL-33 antagonist, an interleukin-4 receptor alpha antagonist, and/or an interleukin-13 receptor antagonist generally). Alternately, a FEV1-PS that is less than a threshold FEV1-PS indicates the subject will not respond adequately to treatment with a standard dosage amount of dupilumab (or a bronchodilator, an IL-33 antagonist, an interleukin-4 receptor alpha antagonist, and/or an interleukin-13 receptor antagonist generally).

The present disclosure also provides methods of classifying a subject having a lung disease or at risk of developing a lung disease who will adequately respond to treatment with dupilumab (or a bronchodilator, an IL-33 antagonist, an interleukin-4 receptor alpha antagonist, and/or an interleukin-13 receptor antagonist generally) by carrying out the methods described herein. The present disclosure also provides methods of selecting a subject having a lung disease or at risk of developing a lung disease for treatment with dupilumab (or a bronchodilator, an IL-33 antagonist, an interleukin-4 receptor alpha antagonist, and/or an interleukin-13 receptor antagonist generally) by carrying out the methods described herein. The present disclosure also provides methods of improving lung disease efficacy by carrying out the methods described herein.

Without being limited by any particular theory, it is believed that the FEV1-PS determined according to the methods presented herein allow for identification of subjects that will likely respond to a therapeutic agent that treats or inhibits a lung disease, including, without limitation, a bronchodilator, an interleukin-4 receptor alpha antagonist and/or an interleukin-13 receptor antagonist, such as dupilumab, or an IL-33 antagonist. Subjects that have a FEV1-PS greater than a threshold FEV1-PS are likely to have a high FEV1 and, thus, respond adequately to treatment with dupilumab. Such subjects can be treated with or continue to be treated with a standard dosage amount of dupilumab. Subjects that have a FEV1-PS less than a threshold FEV1-PS are likely to have a low FEV1 and, thus, not respond adequately to treatment with a standard dosage amount of dupilumab. Such subjects can be treated with a dosage amount of dupilumab that is greater than a standard dosage amount or can be treated with a different therapeutic agent or combination of therapeutic agents. In some embodiments, the favorable therapeutic response to treatment with dupilumab (or a bronchodilator, an IL-33 antagonist, an interleukin-4 receptor alpha antagonist, and/or an interleukin-13 receptor antagonist generally) is measured by an FEV1 in the top quartile.

In some embodiments, the methods disclosed herein are used to select a population of subjects or candidates for clinical trials, e.g., a clinical trial to determine whether a particular treatment or treatment plan is effective against a lung disease, such as asthma or COPD. In some embodiments, the selected candidates or subjects are divided into subgroups based on the identified genetic variants for each subject or candidate, and the method is used to determine whether a particular treatment or treatment plan is effective for a subject having a particular genetic variant or a particular group of genetic variants. For example, the methods described herein can be employed to determine susceptibility of a population of subjects to a particular treatment or treatment plan, wherein the population of subjects is selected based on the genetic variants identified in the subjects. In some embodiments, the desired group is a population comprising subjects or candidates having asthma or COPD. In some embodiments, the selected population of subjects or candidates are responders, i.e., the subjects or candidates are responsive to the treatment or treatment plan.

In some embodiments, the methods described herein further comprise initiating a treatment to the subject. The treatment can comprise a bronchodilator, such as an ICS, a leukotriene modifier, a long-acting beta agonist (LABA), theophylline, combination inhalers that contain both a corticosteroid and a LABA, a short-acting beta agonist such as albuterol, ipratropium, an oral corticosteroid, an intravenous corticosteroid, an allergy shot, an allergy medication, omalizumab, mepolizumab, benralizumab, reslizumab, dupilumab, and itepekimab, or any combination thereof. Initiating a treatment can include devising a treatment plan based on the group, which corresponds to the FEV1-PS calculated for the subject. In some embodiments, a FEV1-PS is predictive of treatment efficacy or of subject's response to a therapeutic regimen. Accordingly, the treatment can be determined or adjusted according to the FEV1-PS.

In some embodiments, the treatment initiation comprises modifying dosage or regimen of a treatment that a subject with a lung disease, such as asthma or COPD, already receives based on a FEV1-PS calculated for the subject. In some embodiments, the treatment initiation comprises substitution of one therapeutic agent with another based on a FEV1-PS. In some embodiments, the treatment initiation comprises starting a regimen of a second therapeutic agent in addition to a first therapeutic agent a subject already receives. In some embodiments, the treatment initiation comprises starting administration of a therapeutic regimen to a previously untreated lung disease subject.

Examples of therapeutic agents that can be used to treat asthma include, but are not limited to, an ICS, a leukotriene modifier, a long-acting beta agonist (LABA), theophylline, combination inhalers that contain both a corticosteroid and a LABA, a short-acting beta agonist such as albuterol, ipratropium, an oral corticosteroid, an intravenous corticosteroid, an allergy shot, an allergy medication, omalizumab, mepolizumab, benralizumab, reslizumab, dupilumab, and itepekimab, or any combination thereof.

In some embodiments, a therapeutic agents that increase FEV1 in asthma can be administered including, but not limited to, OCS, ICS, LABA, LAMA, SABA, SAMA, anti-leukotrienes (such as, montelukast), theophylline, dupilumab, tezepelumab, omalizumab, mepolizumab, benralizumab, and reslizumab, or any combination thereof.

In some embodiments, the therapeutic agent is a bronchodilator, an IL-33 antagonist, an interleukin-4 receptor alpha antagonist, and/or an interleukin-13 receptor antagonist. In some embodiments, an FEV1-PS is predictive of treatment efficacy or of a subject's response to treatment with a bronchodilator, an IL-33 antagonist, an interleukin-4 receptor alpha antagonist, and/or an interleukin-13 receptor antagonist. Accordingly, the bronchodilator, IL-33 antagonist, an interleukin-4 receptor alpha antagonist, and/or an interleukin-13 receptor antagonist treatment can be determined or adjusted according to the FEV1-PS calculated for the subject.

In the methods described herein, the therapeutic agents (e.g., a bronchodilator, an IL-33 antagonist, an interleukin-4 receptor alpha antagonist, and/or an interleukin-13 receptor antagonist) may be capable of inhibiting the activity of the respective protein or other substance in the cell at least to a certain amount. This can be achieved by a direct interaction of the compound with the given protein or substance (“direct inhibition”) or by an interaction of the compound with other proteins or other substances in or outside the cell which leads to an at least partial inhibition of the activity of the protein or substance (“indirect inhibition”). Inhibition of protein activity can also be achieved through suppressing the expression of a target protein. Techniques of inhibiting protein expression include, but not limited to, antisense inhibition, siRNA-mediated inhibition, miRNA mediated inhibition, ribozyme-mediated inhibition, DNA-directed RNA interference (DdRNAi), RNA-directed DNA methylation, transcription activator-like effector nucleases (TALEN)-mediated inhibition, zinc finger nuclease-mediated inhibition, aptamer-mediated inhibition, and CRISPR-mediated inhibition.

In some embodiments, the therapeutic agents (such as bronchodilators, IL-33 antagonists, interleukin-4 receptor alpha antagonists, and/or interleukin-13 receptor antagonists) are inhibitory nucleic acid molecules. In some embodiments, the inhibitory nucleic acid molecule comprises an antisense molecule, a small interfering RNA (siRNA) molecule, or a short hairpin RNA (shRNA) molecule. In some embodiments, the inhibitory nucleic acid molecule comprises an antisense molecule. In some embodiments, the inhibitory nucleic acid molecule comprises an siRNA molecule. In some embodiments, the inhibitory nucleic acid molecule comprises an shRNA molecule. In some embodiments, the therapeutic agents (such as bronchodilators, IL-33 antagonists, interleukin-4 receptor alpha antagonists, and/or interleukin-13 receptor antagonists) are small molecules.

In some embodiments, the IL-33 antagonist is an anti-IL-33 antibody or an antigen binding portion thereof. In some embodiments, the IL-33 antagonist is an anti-IL-33 receptor antagonist. In some embodiments, the interleukin-4 receptor alpha antagonist is an anti-interleukin-4 receptor alpha antibody or an antigen binding fragment thereof. In some embodiments, the anti-interleukin-4 receptor alpha antagonist is an anti-interleukin-4 antibody or an antigen binding fragment thereof. In some embodiments, the interleukin-13 receptor antagonist is an anti-IL-13 receptor antibody. In some embodiments, the interleukin-13 receptor antagonist is an anti-interleukin-13 antibody or antigen-binding fragment thereof.

Antibodies refer to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments, the FRs of the anti-IL-33 antibody (or antigen-binding fragment thereof) or the anti-interleukin-4 receptor alpha (or antigen-binding portion thereof) may be identical to the human germline sequences, or may be naturally or artificially modified. An amino acid consensus sequence may be defined based on a side-by-side analysis of two or more CDRs.

In some embodiments, an “antibody” includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

In some embodiments, the interleukin-4 receptor alpha antagonist specifically binds to human IL-4Rα and comprises a heavy chain variable region (HCVR) comprising SEQ ID NO:1 and a light chain variable region (LCVR) comprising SEQ ID NO:2, a heavy chain complementarity determining region 1 (HCDR1) comprising SEQ ID NO:3, a HCDR2 comprising SEQ ID NO:4, a HCDR3 comprising SEQ ID NO:5, a light chain complementarity determining region 1 (LCDR1) comprising SEQ ID NO:6, a LCDR2 comprising SEQ ID NO:7, and a LCDR3 comprising SEQ ID NO:8. The full-length heavy chain of dupilumab is shown as SEQ ID NO:9 and the full length light chain is shown as SEQ ID NO:10. Human anti-IL-4R antibodies can be generated as described in U.S. Pat. No. 7,608,693.

In some embodiments, the IL-33 antagonist comprises itepekimab. In some embodiments, the interleukin-4 receptor alpha antagonist is dupilumab. In some embodiments, the interleukin-13 receptor antagonist is dupilumab. In some embodiments, the interleukin-4 receptor alpha antagonist and the interleukin-13 receptor antagonist is dupilumab.

In the context of the methods, additional therapeutically active component(s), e.g., any of the agents listed above or derivatives thereof, may be administered just prior to, concurrent with, or shortly after the administration of a bronchodilator, an IL-33 antagonist, an interleukin-4 receptor alpha antagonist, and/or an interleukin-13 receptor antagonist; (for purposes of the present disclosure, such administration regimens are considered the administration of a bronchodilator, an IL-33 antagonist, an interleukin-4 receptor alpha antagonist, and/or an interleukin-13 receptor antagonist “in combination with” an additional therapeutically active component). In some embodiments, an additional therapeutically active component is considered administered “in combination with” a bronchodilator, an IL-33 antagonist, an interleukin-4 receptor alpha antagonist, and/or an interleukin-13 receptor antagonist notwithstanding the fact that the additional therapeutically active component and the bronchodilator, IL-33 antagonist, the interleukin-4 receptor alpha antagonist, and/or the interleukin-13 receptor antagonist are administered by different routes. The present methods include pharmaceutical compositions and methods of use thereof in which a bronchodilator, an IL-33 antagonist, an interleukin-4 receptor alpha antagonist, and/or an interleukin-13 receptor antagonist is co-formulated with one or more of the additional therapeutically active component(s) as described herein.

In any of the embodiments described herein, the lung disease can be any disease in which spirometry is used. In some embodiments, the lung disease is an obstructive lung disease such as, for example, asthma, COPD, and forms of bronchiectasis (such as cystic fibrosis). In some embodiments, the lung disease is a restrictive lung disease such as, for example, interstitial lung diseases including idiopathic pulmonary fibrosis. In any of the embodiments described herein, the lung disease can be a disease including, but not limited to, asthma, COPD, pulmonary fibrosis, chronic bronchitis, emphysema, acute bronchitis, cystic fibrosis, a bacterial lung infection, a mycobacterial infection, pneumonia, tuberculosis caused by, without limitation, Mycobacterium tuberculosis, pulmonary edema, lung cancer, acute respiratory distress syndrome (ARDS) including, without limitation, ARDS related to COVID, pneumoconiosis, lung damage caused by a chemical, biological, or radio-nuclear (CBRN) agent, black lung disease relating from exposure to coal dust, and asbestosis relating to exposure to asbestos. In some embodiments, the asthma can be, without limitation, mild asthma, moderate asthma, severe asthma, eosinophilic asthma with or without changes to immunoglobulin E levels, or oral corticosteroid-dependent asthma. In some embodiments, asthma exacerbation can be annualized asthma exacerbation. In some embodiments, the lung disease is asthma. In some embodiments, the lung disease is COPD.

In some embodiments, a subject who is treatable by the methods of the present disclosure has had a lung disease within the past 1, 2, 3, 4, 5, 6, 7, 8, 9, 10, 11, 12, 13, 14, 15, 16, 17, or 18 months. Subjects who are treatable by the methods of the present disclosure include subjects that have been hospitalized with asthma-related or COPD-related symptoms and subjects that currently are hospitalized.

Assessments using large numbers of genetic variants offers the advantage of increased predictive power. In some embodiments, the plurality of genetic variants comprises a single nucleotide polymorphism (SNP), an insertion, a deletion, a structural variant, or a copy-number variation, or any combination thereof. In some embodiments, one or more of the genetic variants is a SNP. In some embodiments, one or more of the genetic variants is an insertion. In some embodiments, one or more of the genetic variants is a deletion. In some embodiments, one or more of the genetic variants is a structural variant. In some embodiments, one or more of the genetic variants is a copy-number variation.

In some embodiments, the subject may be selected on the basis of FEV1-PS, wherein the FEV1-PS comprises an aggregate (or a weighted aggregate) of a plurality of genetic variants associated with a pulmonary metric, such as pre-bronchodilator FEV1, and is calculated using at least about 150, at least about 500, at least about 1,000, at least about 10,000, at least about 50,000, at least about 100,000, at least about 250,000, at least about 500,000, at least about 750,000, at least about 1,000,000, at least about 1,500,000, at least about 2,000,000, at least about 2,500,000, at least about 3,000,000, at least about 5,000,000, or at least about 10,000,000 genetic variants associated with the pulmonary metric, such as pre-bronchodilator FEV1.

In some embodiments, the present disclosure provides methods of determining an FEV1-PS in a subject, the method comprising identifying whether at least about 150, at least about 500, at least about 1,000, at least about 10,000, at least about 50,000, at least about 100,000, at least about 250,000, at least about 500,000, at least about 750,000, at least about 1,000,000, at least about 1,500,000, at least about 2,000,000, at least about 2,500,000, at least about 3,000,000, at least about 5,000,000, or at least about 10,000,000 genetic variants associated with a pulmonary metric, such as pre-bronchodilator FEV1, are present in a biological sample from the subject; wherein the presence of an allele associated with a pulmonary metric, such as pre-bronchodilator FEV1, increases the FEV1-PS.

In some embodiments, the disclosure provides a method of determining FEV1-PS in a subject comprising identifying whether genetic variants associated with a pulmonary metric, such as pre-bronchodilator FEV1, are present in a biological sample from the subject and calculating a FEV1-PS for the subject based on the identified genetic variants, wherein the FEV1-PS is calculated by summing the weighted score associated with each genetic variant identified. The number of identified genetic variants can be at least about 150, at least about 500, at least about 1,000, at least about 10,000, at least about 50,000, at least about 100,000, at least about 250,000, at least about 500,000, at least about 750,000, at least about 1,000,000, at least about 1,500,000, at least about 2,000,000, at least about 2,500,000, at least about 3,000,000, at least about 5,000,000, or at least about 10,000,000 genetic variants.

In some embodiments, the disclosure provides a method of determining FEV1-PS in a subject comprising identifying whether the genetic variants associated with a pulmonary metric, such as pre-bronchodilator FEV1, are present in a biological sample from the subject, wherein the identifying comprises measuring the presence of the at least about 150, at least about 500, at least about 1,000, at least about 10,000, at least about 50,000, at least about 100,000, at least about 250,000, at least about 500,000, at least about 750,000, at least about 1,000,000, at least about 1,500,000, at least about 2,000,000, at least about 2,500,000, at least about 3,000,000, at least about 5,000,000, or at least about 10,000,000 genetic variants.

In some embodiments, the disclosure provides a method of determining a FEV1-PS in a subject comprising selecting at least about 2 genetic variants, at least about 10 genetic variants, at least about 25 genetic variants, at least about 50 genetic variants, at least about 100 genetic variants, at least about 150 genetic variants, at least about 200 genetic variants, at least about 500 genetic variants, at least about 1000 genetic variants, at least about 2000 genetic variants, at least about 5000 genetic variants, at least about 10,000 genetic variants, at least about 20,000 genetic variants, at least about 50,000 genetic variants, at least about 75,000 genetic variants, at least about 100,000 genetic variants, at least about 500,000 genetic variants, at least about 1,000,000 genetic variants, at least about 2,000,000 genetic variants, at least about 2,500,000 genetic variants, at least about 3,000,000 genetic variants, at least about 5,000,000 genetic variants, or at least about 10,000,000 genetic variants associated with a pulmonary metric, such as pre-bronchodilator FEV1; identifying whether the genetic variants are present in a biological sample from the subject; and calculating the FEV1-PS based on the presence of the genetic variants.

In some embodiments, the plurality of genetic variants in the FEV1-PS determination comprises at least 2 genetic variants associated with a pulmonary metric, such as pre-bronchodilator FEV1. In some embodiments, the plurality of genetic variants in the FEV1-PS determination comprises at least 10 genetic variants associated with a pulmonary metric, such as pre-bronchodilator FEV1. In some embodiments, the plurality of genetic variants in the FEV1-PS determination comprises at least 25 genetic variants associated with a pulmonary metric, such as pre-bronchodilator FEV1. In some embodiments, the plurality of genetic variants in the FEV1-PS determination comprises at least 50 genetic variants associated with a pulmonary metric, such as pre-bronchodilator FEV1. In some embodiments, the plurality of genetic variants in the FEV1-PS determination comprises at least 100 genetic variants associated with a pulmonary metric, such as pre-bronchodilator FEV1. In some embodiments, the plurality of genetic variants in the FEV1-PS determination comprises at least 150 genetic variants associated with a pulmonary metric, such as pre-bronchodilator FEV1. In some embodiments, the plurality of genetic variants in the FEV1-PS determination comprises at least 1,000 genetic variants associated with a pulmonary metric, such as pre-bronchodilator FEV1. In some embodiments, the plurality of genetic variants in the FEV1-PS determination comprises at least 10,000 genetic variants associated with a pulmonary metric, such as pre-bronchodilator FEV1. In some embodiments, the plurality of genetic variants in the FEV1-PS determination comprises at least 100,000 genetic variants associated with a pulmonary metric, such as pre-bronchodilator FEV1. In some embodiments, the plurality of genetic variants in the FEV1-PS determination comprises at least 1,000,000 genetic variants associated with a pulmonary metric, such as pre-bronchodilator FEV1. In some embodiments, the plurality of genetic variants in the FEV1-PS determination comprises at least 2,000,000 genetic variants associated with a pulmonary metric, such as pre-bronchodilator FEV1. In some embodiments, the plurality of genetic variants in the FEV1-PS determination comprises at least 2,500,000 genetic variants associated with a pulmonary metric, such as pre-bronchodilator FEV1. In some embodiments, the plurality of genetic variants in the FEV1-PS determination comprises at least 3,000,000 genetic variants associated with a pulmonary metric, such as pre-bronchodilator FEV1. In some embodiments, the plurality of genetic variants in the FEV1-PS determination comprises at least 5,000,000 genetic variants associated with a pulmonary metric, such as pre-bronchodilator FEV1. In some embodiments, the plurality of genetic variants in the FEV1-PS determination comprises at least 10,000,000 genetic variants associated with a pulmonary metric, such as pre-bronchodilator FEV1.

In some embodiments, the disclosure provides methods for selecting subjects or candidates for administration of a therapeutic agent that treats or inhibits a lung disease, including but not limited to an IL-33 antagonist, an interleukin-4 receptor alpha antagonist, and/or an interleukin-13 receptor antagonist comprising identifying whether at least about 150, at least about 500, at least about 1,000, at least about 10,000, at least about 50,000, at least about 100,000, at least about 250,000, at least about 500,000, at least about 750,000, at least about 1,000,000, at least about 1,500,000, at least about 2,000,000, at least about 2,500,000, at least about 3,000,000, at least about 5,000,000, or at least about 10,000,000 genetic variants are present in a biological sample from each subject or candidate; calculating a FEV1-PS for each subject or candidate based on the identified genetic variants; and selecting the subjects or candidates for administration of a therapeutic agent that treats or inhibits a lung disease, including without limitation, an IL-33 antagonist, such as itepekimab, or a interleukin-4 receptor alpha antagonist or an interleukin-13 receptor antagonist, such as dupilumab.

In some embodiments, the disclosure provides a method for selecting a population of subjects or candidates for administration of a therapeutic agent that treats or inhibits a lung disease, including but not limited to an IL-33 antagonist, a interleukin-4 receptor alpha antagonist, and/or an interleukin-13 receptor antagonist comprising identifying whether at least about 150, at least about 500, at least about 1,000, at least about 10,000, at least about 50,000, at least about 100,000, at least about 250,000, at least about 500,000, at least about 750,000, at least about 1,000,000, at least about 1,500,000, at least about 2,000,000, at least about 2,500,000, at least about 3,000,000, at least about 5,000,000, or at least about 10,000,000 genetic variants are present in a biological sample from each subject or candidate; calculating a FEV1-PS for each subject or candidate based on the identified genetic variants; and selecting the subjects or candidates for administration of a therapeutic agent that treats or inhibits a lung disease, including but not limited to a bronchodilator, an IL-33 antagonist, an interleukin-4 receptor alpha antagonist, and/or an interleukin-13 receptor antagonist.

In some embodiments, the number of identified genetic variants is at least 2 genetic variants associated with a pulmonary metric, such as pre-bronchodilator FEV1. In some embodiments, the number of identified genetic variants is at least 10 genetic variants associated with a pulmonary metric, such as pre-bronchodilator FEV1. In some embodiments, the number of identified genetic variants is at least 25 genetic variants associated with a pulmonary metric, such as pre-bronchodilator FEV1. In some embodiments, the number of identified genetic variants is at least 50 genetic variants associated with a pulmonary metric, such as pre-bronchodilator FEV1. In some embodiments, the number of identified genetic variants is at least 100 genetic variants associated with a pulmonary metric, such as pre-bronchodilator FEV1. In some embodiments, the number of identified genetic variants is at least 150 genetic variants associated with a pulmonary metric, such as pre-bronchodilator FEV1. In some embodiments, the number of identified genetic variants is at least 500 genetic variants associated with a pulmonary metric, such as pre-bronchodilator FEV1. In some embodiments, the number of identified genetic variants is at least 1,000 genetic variants associated with a pulmonary metric, such as pre-bronchodilator FEV1. In some embodiments, the number of identified genetic variants is at least 2,000 genetic variants associated with a pulmonary metric, such as pre-bronchodilator FEV1. In some embodiments, the number of identified genetic variants is at least 5,000 genetic variants associated with a pulmonary metric, such as pre-bronchodilator FEV1. In some embodiments, the number of identified genetic variants is at least 10,000 genetic variants associated with a pulmonary metric, such as pre-bronchodilator FEV1. In some embodiments, the number of identified genetic variants is at least 20,000 genetic variants associated with a pulmonary metric, such as pre-bronchodilator FEV1. In some embodiments, the number of identified genetic variants is at least 50,000 genetic variants associated with a pulmonary metric, such as pre-bronchodilator FEV1. In some embodiments, the number of identified genetic variants is at least 75,000 genetic variants associated with a pulmonary metric, such as pre-bronchodilator FEV1. In some embodiments, the number of identified genetic variants is at least 100,000 genetic variants associated with a pulmonary metric, such as pre-bronchodilator FEV1. In some embodiments, the number of identified genetic variants is at least 500,000 genetic variants associated with a pulmonary metric, such as pre-bronchodilator FEV1. In some embodiments, the number of identified genetic variants is at least 1,000,000 genetic variants associated with a pulmonary metric, such as pre-bronchodilator FEV1. In some embodiments, the number of identified genetic variants is at least 2,000,000 genetic variants associated with a pulmonary metric, such as pre-bronchodilator FEV1. In some embodiments, the number of identified genetic variants is at least 3,000,000 genetic variants associated with a pulmonary metric, such as pre-bronchodilator FEV1. In some embodiments, the number of identified genetic variants is at least 5,000,000 genetic variants associated with a pulmonary metric, such as pre-bronchodilator FEV1. In some embodiments, the number of identified genetic variants is at least 10,000,000 genetic variants associated with a pulmonary metric, such as pre-bronchodilator FEV1.

In some embodiments, the genetic variants include any one or more of the variants listed in Table 1 (variant id is according to GRCh38/hg38 human genome assembly coordinates).

TABLE 1

| | | |
|---|---|---|
| variantjd | variantjd | variantjd |
| 2:18106357:T:C | 16:3563125:C:T | 3:49505682:T:G |
| 2:157668714:C:T | 17:7463300:T:C | 3:53638444:T:C |
| 2:178498958:C:T | 17:35745536:G:A | 3:71591625:A:G |
| 3:168992055:G:T | 19:45790878:G:A | 3:96120805:G:A |
| 4:55145982:A:G | 20:35437976:G:A | 3:134622008:A:G |
| 4:144516167:A:G | 1:19354779:G:T | 3:169577648:A:G |
| 5:122695016:G:A | 1:22331992:G:A | 4:105893145:A:G |
| 6:26200449:A:G | 1:395272O6:T:G | 4:123966904:G:A |
| 6:108946847:C:T | 1:45555958:C:T | 4:169087957:G:A |
| 9:18009673:G:T | 1:77811292:G:A | 5:43484155:C:T |
| 9:95606479:T:C | 1:155194689:A:C | 5:43976060:A:G |
| 10:12235993:C:T | 1:178932O55:A:G | 5:53018217:C:T |
| 10:76555466:G:A | l:204457167:A:G | 5:54148668:C:T |
| 12:55996852:A:G | l:221030957:A:C | 5:72984117:T:G |
| 16:28814728:C:T | 1:221559914:C:T | 5:77603795:T:C |
| 16:69522812:C:T | 1:239725457:C:T | 5:79261418:A:G |
| 18:10078074:G:A | 2:18504178:C:T | 5:127980639:A:G |
| 2:217818431:A:G | 2:18544473:G:A | 5:129431382:A:G |
| 4:3494214:T:C | 2:62975862:T:C | 5:142389810:G:A |
| 4:105897896:G:A | 2:65849693:G:A | 5:148476959:G:A |
| 4:144737912:T:C | 2:150249342:C:A | 5:148823673:T:C |
| 5:148827322:C:T | 2:198836007:A:C | 5:149020682:A:G |
| 10:484835:G:A | 2:205701018:G:A | 5:149272739:G:T |
| 10:73816205:T:C | 2:209445813:T:G | 5:157493748:G:T |
| 12:2799164:C:A | 2:238532667:C:A | 5:158937441:G:A |
| 12:110345436:G:A | 2:238656934:C:T | 6:7779496:G:T |
| 6:75724642:T:C | 3:11626240:C:T | 6:22056694:A:C |
| 6:84268764:T:C | 3:30689380:G:A | 6:34179034:G:A |
| 6:105034959:G:A | 9:98870572:G:A | 6:35443314:G:A |
| 6:109332622:C:T | 9:136205927:A:C | 14:102073442:G:A |
| 6:141440196:C:T | 10:13628971:A:G | 15:40962955:C:T |
| 6:142481900:C:T | 10:51012717:G:A | 15:61116661:A:G |
| 6:152271967:A:G | 10:75899975:G:A | 15:71395057:C:T |
| 6:154960359:T:C | 10:121272654:G:A | 15:78533838:G:A |
| 6:169216017:C:T | 11:13149689:C:T | 15:83617585:A:C |
| 7:7216859:G:A | ll:66810462:A:G | 16:9988317:A:G |
| 7:14796854:G:T | 11:69646414:C:T | 16:14637534:C:A |
| 7:18304472:G:A | 11:1261396O5:T:C | 16:78125512:C:T |
| 7:18478629:G:A | 11:132117385:C:T | 16:88746652:C:T |
| 7:23444609:G:A | 12:27857474:T:C | 16:89676592:G:A |
| 7:26824071:C:A | 12:28435309:T:C | 17:12405813:T:G |
| 7:28171041:T:C | 12:28687959:A:G | 17:38691460:G:A |
| 7:46408920:T:C | 12:65511346:C:T | 17:48470019:A:G |
| 7:156334552:G:A | 12:65980807:T:C | 17:75529589:T:G |
| 8:11898295:T:C | 12:83575321:A:G | 18:8801353:A:G |
| 8:13216899:A:C | 12:114235616:G:A | 18:22436336:T:G |
| 8:69466261:A:G | 12:115063322:G:A | 18:53236679:T:C |
| 8:102119072:T:C | 13:49803774:A:C | 18:55840828:G:A |
| 8:108259777:T:C | 13:71112040:A:G | 19:5897836:T:G |
| 8:129016354:G:A | 13:79893100:C:T | 20:32454373:C:T |
| 9:1555814:G:A | 14:26712315:A:G | 20:46874226:C:T |
| 9:4144772:C:T | 14:37070031:T:C | 20:51644472:A:G |
| 9:23579107:T:G | 14:74350715:A:G | 21:34709150:G:T |
| 9:81140293:T:C | 14:92548584:G:A | 22:17954616:A:G |
| | | 22:30201679:A:G |

As an exemplary method, a FEV1-PS can be determined from, for example, data obtained from a GWAS related to a pulmonary metric, such as pre-bronchodilator FEV1. For example, in a representative hypothetical GWAS, a GWAS may have identified four genetic variants associated with a pulmonary metric, such as pre-bronchodilator FEV1. Each of the genetic variants may be associated with one or more genes. A value, such as an Odds Ratio, can be calculated for each individual genetic variant. A particular subject's FEV1-PS can be determined by multiplying the log value of the individual Odds Ratio for each variant by the Number Effect Alleles (which is the number of copies of the genetic variant in the genome; i.e., either 0, 1, or 2), and then summing the resultant values. This type of determination can be described by the following Table 2.

TABLE 2

| Gene | Variant | Effect | Odds Ratio | Number | Log(OR) x Number |
|---|---|---|---|---|---|
| | rsID | Allele | (OR) | Effect | Effect Alleles |
| | | | | Alleles | |
| A | rsOOOOOl | T | 2.14 | 1 | 0.761 |
| B | rsOOOOO2 | A | 1.85 | 0 | 0.000 |
| C | rs000003 | A | 1.36 | 0 | 0.000 |
| D | rs000004 | C | 1.28 | 1 | 0.247 |

| | |
|---|---|
| Total Score | 10.910 |

Thus, the subject's FEV1-PS is the sum of the individual values in the last column of the Table taking into consideration any number of genetic variants associated with a pulmonary metric, such as pre-bronchodilator FEV1. This simplified methodology for determining a subject's FEV1-PS is for exemplary purposes only and shall not be construed to be limiting in any manner. The FEV1-PS in the above table is a weighted score because each genetic variant may carry a different weight depending on the particular Odds Ratio and the Number Effect Alleles value.

In some embodiments, the disclosure provides methods of assigning a risk group to a subject. The methods comprise identifying whether genetic variants are present in a biological sample from the subject, calculating a FEV1-PS for the subject based on the identified genetic variants, and assigning the subject to a risk group based on the FEV1-PS. The FEV1-PS may be divided into quintiles, e.g., top quintile, top-intermediate quintile, intermediate quintile, intermediate-bottom quartile, and bottom quintile, wherein the top quintile of FEV1-PSs correspond the highest genetic risk group and the bottom quintile of FEV1-PSs correspond to the lowest genetic risk group. The number of identified genetic variants can be at least about 150, at least about 500, at least about 1,000, at least about 10,000, at least about 50,000, at least about 100,000, at least about 250,000, at least about 500,000, at least about 750,000, at least about 1,000,000, at least about 1,500,000, at least about 2,000,000, at least about 2,500,000, at least about 3,000,000, at least about 5,000,000, or at least about 10,000,000 genetic variants. In some embodiments, the threshold FEV1-PS is the top quartile within a reference population. In some embodiments, the threshold FEV1-PS is the top quintile within a reference population. In some embodiments, the threshold FEV1-PS is the top decile within a reference population.

In some embodiments, risk assessments comprise the highest weighted FEV1-PS scores, including, but not limited to the top 50%, 55%, 60%, 70%, 80%, 90%, or 95% of FEV1-PS scores from a subject population. In some embodiments of the disclosure, the threshold FEV1-PS is a value within the top 50th, 55th, 60th, 70th, 80th, 90th, or 95th percentile of the FEV1-PS value. In some embodiments of the disclosure, the threshold FEV1-PS is a value within the top 50th percentile of the FEV1-PS value. In some embodiments of the disclosure, the threshold FEV1-PS is a value within the top 55th percentile of the FEV1-PS value. In some embodiments of the disclosure, the threshold FEV1-PS is a value within the top 60th percentile of the FEV1-PS value. In some embodiments of the disclosure, the threshold FEV1-PS is a value within the top 65th percentile of the FEV1-PS value. In some embodiments of the disclosure, the threshold FEV1-PS is a value within the top 70th percentile of the FEV1-PS value. In some embodiments of the disclosure, the threshold FEV1-PS is a value within the top 75th percentile of the FEV1-PS value. In some embodiments of the disclosure, the threshold FEV1-PS is a value within the top 80th percentile of the FEV1-PS value. In some embodiments of the disclosure, the threshold FEV1-PS is a value within the top 85th percentile of the FEV1-PS value. In some embodiments of the disclosure, the threshold FEV1-PS is a value within the top 90th percentile of the FEV1-PS value. In some embodiments of the disclosure, the threshold FEV1-PS is a value within the top 95th percentile of the FEV1-PS value.

In some embodiments, the identified genetic variants comprise the highest genetic variants or genetic variants with a weighted score in the top 10th, top 20th, top 30th, top 40th, or top 50th percentile. In some embodiments, the identified genetic variants comprise the highest genetic variants or genetic variants with a weighted score in the top 10th percentile. In some embodiments, the identified genetic variants comprise the highest genetic variants or genetic variants with a weighted score in the top 20th percentile. In some embodiments, the identified genetic variants comprise the highest genetic variants or genetic variants with a weighted score in the top 30th percentile. In some embodiments, the identified genetic variants comprise the highest genetic variants or genetic variants with a weighted score in the top 40th percentile. In some embodiments, the identified genetic variants comprise the highest genetic variants or genetic variants with a weighted score in the top 50th percentile.

In some embodiments, the identified genetic variants comprise the genetic variants having association with asthma in the top 10%, top 20%, top 30%, top 40%, or top 50% of a p-value range. In some embodiments, the identified genetic variants comprise the genetic variants having association with asthma in the top 10% of a p-value range. In some embodiments, the identified genetic variants comprise the genetic variants having association with asthma in the top 20% of a p-value range. In some embodiments, the identified genetic variants comprise the genetic variants having association with asthma in the top 30% of a p-value range. In some embodiments, the identified genetic variants comprise the genetic variants having association with asthma in the top 40% of a p-value range. In some embodiments, the identified genetic variants comprise the genetic variants having association with asthma in the top 50% of a p-value range.

In some embodiments, each of the identified genetic variants comprise the genetic variants with a p-value of not larger than about 10⁻¹, about 10⁻², about 10⁻³, about 10⁻⁴, about 10⁻⁵, about 10⁻⁶, about 10⁻⁷ 10⁻⁸, about 10⁻⁸, about 10⁻¹⁰, about 10⁻¹¹, about 10⁻¹², about 10⁻¹³, about 10⁻¹⁴, about or 10⁻¹⁵. In some embodiments, the identified genetic variants comprise the genetic variants with a p-value of less than 5×10⁻⁸.

In some embodiments, the identified genetic variants comprise genetic variants having association with subjects as compared to the rest of the reference population with odds ratio (OR) of about 1.0 or greater, about 1.5 or greater, about 1.75 or greater, about 2.0 or greater, or about 2.25 or greater for the top up to 50% of the distribution; or about 1.5 or greater, about 1.75 or greater, about 2.0 or greater, about 2.25 or greater, about 2.5 or greater, or about 2.75 or greater. In some embodiments, the OR may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, or from about 6.5 to about 7.0. In some embodiments, the subjects comprise subjects having FEV1-PS scores in the top decile, quintile, or tertile in a reference population.

In some embodiments the subjects are selected based on FEV1-PS alone. For example, if a subject or a candidate subject that has a FEV1-PS above or equal to a pre-determined threshold, the subject is selected for initiating treatment or a candidate is included in the clinical trial. In some embodiments, the threshold for treatment initiation or clinical trial inclusion is determined in relative terms. For example, in some embodiments, the threshold FEV1-PS score is the top 50% within a reference population. In some embodiments, embodiments the threshold FEV1-PS score is the top 40% within a reference population. In some embodiments, the threshold FEV1-PS score is the top 30% within a reference population. In some embodiments, the threshold FEV1-PS score is the top 25% within a reference population. In some embodiments, the threshold FEV1-PS score is the top 20% within a reference population. In some embodiments, the threshold FEV1-PS score is the top 15% within a reference population. In some embodiments, the threshold FEV1-PS score is the top 10% (decile) within a reference population. In some embodiments, the threshold FEV1-PS score is the top 5% within a reference population.

In some embodiments, the identified genetic variants comprise the genetic variants having the highest genetic variant performance in the reference population. In some embodiments, genetic variant performance is calculated with respect to a FEV1-PS based on statistical significance, strength of association, and/or a probability distribution.

In some embodiments, the reference population for determination of relative FEV1-PS is at least about 100 subjects. In some embodiments, the reference population for determination of relative FEV1-PS is at least about 200 subjects. In some embodiments, the reference population for determination of relative FEV1-PS is at least about 500 subjects. In some embodiments, the reference population for determination of relative FEV1-PS is at least about 1,000 subjects. In some embodiments, the reference population for determination of relative FEV1-PS is at least about 3,000 subjects. In some embodiments, the reference population for determination of relative FEV1-PS is at least about 5,000 subjects. In some embodiments, the reference population for determination of relative FEV1-PS is at least about 7,500 subjects. In some embodiments, the reference population for determination of relative FEV1-PS is at least about 10,000 subjects. In some embodiments, the reference population for determination of relative FEV1-PS is at least about 12,000 subjects. In some embodiments, the reference population for determination of relative FEV1-PS is at least about 15,000 subjects. In some embodiments, the reference population for determination of relative FEV1-PS is at least about 20,000 subjects. In some embodiments, the reference population for determination of relative FEV1-PS is at least about 30,000 subjects. In some embodiments, the reference population for determination of relative FEV1-PS is at least about 50,000 subjects. In some embodiments, the reference population for determination of relative FEV1-PS is at least about 70,000 subjects. In some embodiments, the reference population for determination of relative FEV1-PS is at least about 100,000 subjects.

In some embodiments, the reference population is enriched for members of an ancestry group. In some embodiments, the ancestry group is derived from a principal component analysis of ancestry. In some embodiments, the ancestry group comprises a European ancestry group, an African ancestry group, an admixed American ancestry group, an East Asian ancestry group, or a South Asian ancestry group. In some embodiments the ancestry group is European. In some embodiments the ancestry group is African. In some embodiments the ancestry group is admixed American. In some embodiments the ancestry group is East Asian. In some embodiments the ancestry group is South Asian. In some embodiments the ancestry group is any mixture of any two or more of the European, African, admixed American, East Asian, and South Asian populations. In some embodiments, a member of the ancestry group has self-reported membership of the ancestry group. In some embodiments, a member of the ancestry group is assigned to the ancestry group based upon genetic testing to the ancestry group.

In some embodiments, the plurality of genetic variants is determined by calculating a genetic variant performance in the reference population and selecting the highest performing genetic variants. Numerous methodologies can be used to calculate a FEV1-PS. In some embodiments, the genetic variant performance is calculated with respect to a strength of association and/or a probability distribution. In some embodiments, the FEV1-PS is calculated using an LDPred or an SBayesR method, or any other available method. In some embodiments, genetic variant scores are calculated using PRS calculation methodologies, such as the LDPred method (or variations and/or versions thereof). LDPred is a Bayesian approach to calculate a posterior mean effect for all variants based on a prior (effect size in the prior genome-wide association study) and subsequent shrinkage based on linkage disequilibrium. LDPred creates a PRS using genome-wide variation with weights derived from a set of GWAS summary statistics. See, Vilhjálnnsson et al., Am. J. Hum. Genet., 2015, 97, 576-92. In some embodiments, alternate approaches for calculating genetic variant scores may be used, including SBayesR (Lloyd-Jones, LR, world wide web at “biorxiv.org/content/biorxiv/early/2019/01/17/522961.full.pdf”), Pruning and Thresholding (P&T) (Purcell, Nature, 2009, 460, 748-752), and conditional and joint analysis (COJO) (Yang et al., Nat. Genet., 2012, 44, 369-375). SBayesR is a Bayesian approach is similar to LDPred but allows for more flexibility in the posterior mean effects. Pruning and Thresholding (P&T) requires that a minimum p-value threshold (p-value associated with the variant from the source data file) and r² threshold (measure of linkage disequilibrium (LD)) between variants be specified. P&T identifies the variant with the smallest p-value in each region and then “clumps” under that variant all other variants in the region with an r² value that is larger than the specified r². In the PRS, the index variant represents all the variants in the clump (only the index variant is included in the PRS with all other variants are excluded). COJO is similar conceptually to P&T but incorporates additional variants in a given LD block into the score if they demonstrate independent contribution to disease risk after conditioning on the index variant. In some embodiments, a subject's race and size is taken into accounting when determining the FEV1-PS.

In some embodiments, genetic variant performance is calculated using the LDPred method, wherein the p value is from about 0.0001 to about 0.5. In some embodiments, genetic variant performance is calculated using the LDPred method, wherein the p value is about 0.5. In some embodiments, genetic variant performance is calculated using the LDPred method, wherein the p value is about 0.1. In some embodiments, genetic variant performance is calculated using the LDPred method, wherein the p value is about 0.05. In some embodiments, genetic variant performance is calculated using the LDPred method, wherein the p value is about 0.01. In some embodiments, genetic variant performance is calculated using the LDPred method, wherein the p value is about 0.005. In some embodiments, genetic variant performance is calculated using the LDpred method, wherein the p value is about 0.001. In some embodiments, genetic variant performance is calculated using the LDPred method, wherein the p value is about 0.0005. In some embodiments, genetic variant performance is calculated using the LDPred method, wherein the ρ value is about 0.0001. In some embodiments, a fraction of causal markers (ρ) is set at 0.001 and the plurality of genetic variants comprises at least 2 genetic variants. In some embodiments, the FEV1-PS is calculated using a pruning and thresholding method. In some embodiments, a p-value threshold is 5×10⁻⁸ and an r² value is 0.2. In some embodiments, a p-value threshold is 5×10⁻² and an r² value is 0.8.

In some embodiments, the methods further comprise an initial step of obtaining a biological sample from the subject. A biological sample may contain whole cells live cells and/or cell debris. The biological sample may contain (or be derived from) a “bodily fluid”. The present disclosure encompasses embodiments wherein the bodily fluid is selected from amniotic fluid, aqueous humor, vitreous humor, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, and cell cultures from bodily fluids. Bodily fluids may be obtained from a mammalian organism, for example by venapuncture, or other collecting or sampling procedures. In some embodiments, the FEV1-PS is determined from a biological sample obtained from the subject, wherein the biological sample comprises blood, semen, saliva, urine, feces, hair, teeth, bone, tissue, a swab from a cheek, or a cell. In some embodiments, the biological sample comprises blood.

In any of the embodiments described herein, a subject who adequately responds to treatment with dupilumab, or any bronchodilator, IL-33 antagonist, an interleukin-4 receptor alpha antagonist, and/or an interleukin-13 receptor antagonist, has or will experience a desired biological response, such as a therapeutic and/or prophylactic effect. In some embodiments, a therapeutic effect comprises one or more of a decrease/reduction in the severity of the lung disease, a decrease/reduction in symptoms of the lung disease-related effects, delaying the onset of symptoms of the lung disease-related effects, reducing the severity of symptoms of the lung disease-related effects, reducing the severity of an acute episode, reducing the number of symptoms of the lung disease-related effects, reducing the latency of symptoms of the lung disease-related effects, an amelioration of symptoms of the lung disease-related effects, reducing secondary symptoms, preventing relapse to the lung disease, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of the lung disease development/progression (such as, for example, a complete or partial avoidance/inhibition or a delay), and an increased survival time of the affected host animal, following administration of a therapeutic protocol. Treatment of the lung disease encompasses the treatment of subjects already diagnosed as having any form of the lung disease at any clinical stage or manifestation, the delay of the onset or evolution or aggravation or deterioration of the symptoms or signs of the lung disease, and/or preventing and/or reducing the severity of the lung disease.

In any of the embodiments described herein, the methods can further comprise prescribing a bronchodilator, an IL-33 antagonist, an interleukin-4 receptor alpha antagonist, and/or an interleukin-13 receptor antagonist.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: Generation of Therapeutic Polygenic Scores (FEV1-PS)

### Datasets:

A genome-wide FEV1-PS for a European population was generated. The FEV1-PS comprised 2.27 million variants. Individual SNP contribution was estimated from the GWAS using the SBayesR method.

### PRS Algorithm Selection:

The LDPred approach to generating FEV1-PS was used. LDPred is a Bayesian approach to PRS development that calculates a posterior mean effect (adjusted effect size) for all variants based on a prior and LD information from a reference panel. Heuristically, the effect sizes generated from LDPred differs from P&T in that LDPred jointly models the effect size and variance of each marker, incorporating the LD structure when shrinking the effect sizes. Adjustment or shrinkage of variant weights is based not only on magnitude of variant association with disease but also linkage disequilibrium (LD) between variants. For the LDPred approach, 1000 Genomes phase 3 version 5 data was used for the LD reference panel.

### PRS Calculation:

From the LDPred approach, a set of variants and their respective weights were generated. In the case of LDPred, the variant weights were the adjusted log odds ratio (posterior mean). After generation of weights, the process for calculating and normalizing scores was identical. For a set of i=1, . . . , M variants in j=1, . . . N patients, the PRS for patient j was calculated by: PRSᵢⱼ=Σᵢ₌₁^{M}(Bᵢxᵢⱼ), where Bᵢ is the log odds ratio for variant i and xᵢⱼ is the number of variant alleles carried by patient j at variant i (for imputed variants, the allele dosage for variant i). Scores were standardized to ^{˜}N(0,1) by subtracting the mean PRS and dividing by the FEV1-PS standard deviation within each ancestry group.

### Testing and Validating PRS Algorithms:

For each set of LDPred tuning parameter, a PRS was calculated and a logistic regression run with the composite endpoint as the dependent variable and PRS, age, sex, genotyping array (UK Biobank only), and ancestry covariates as independent variables. The odds ratio (OR) per PRS standard deviation (SD) and area under the curve (AUC) were reported for each model. LDPred with ρ=0.001 demonstrated the best performance and was used in the primary analysis.

Summary Bayesian Mutilple Regression (SBayesR) is another Bayesian based PRS approach that calculates a posterior mean effect (adjusted effect size) for all variants based on a prior (effect size reported in summary source data) and LD information from a reference panel. In comparison to LDPred, which makes use of point normal distribution as the prior distribution, SBayesR utilizes a mixture normal distribution as the prior distribution to estimate the joint effect of the weights across the whole genome. SBayesR calculates the posterior mean effect size by using an MCMC Gibbs sampler.

### Selection of a Threshold for Defining High Risk:

Genetic high risk was defined as patients within the 25th, 50th, 75th, and 100th percentile of the distribution of the FEV1-PS. This threshold was selected in a post hoc analysis, which evaluated high FEV1-PS thresholds ranging from the 25th, 50th, 75th, and 100th percentile.

### Statistical Analysis:

Baseline disease and medical history characteristics were analyzed to assess the distribution of asthma risk factors by genetic risk status, high (>percentile threshold) vs lower percentile threshold). Continuous baseline characteristics were compared using a t-test, and binary or categorical characteristics were tested with a chi-square or Fisher's exact test.

### Results:

The PRS in this study was developed using GWAS data from individuals of European ancestry. As GWAS data becomes available in more diverse populations, FEV1-PS will likely improve over time for non-European populations as well.

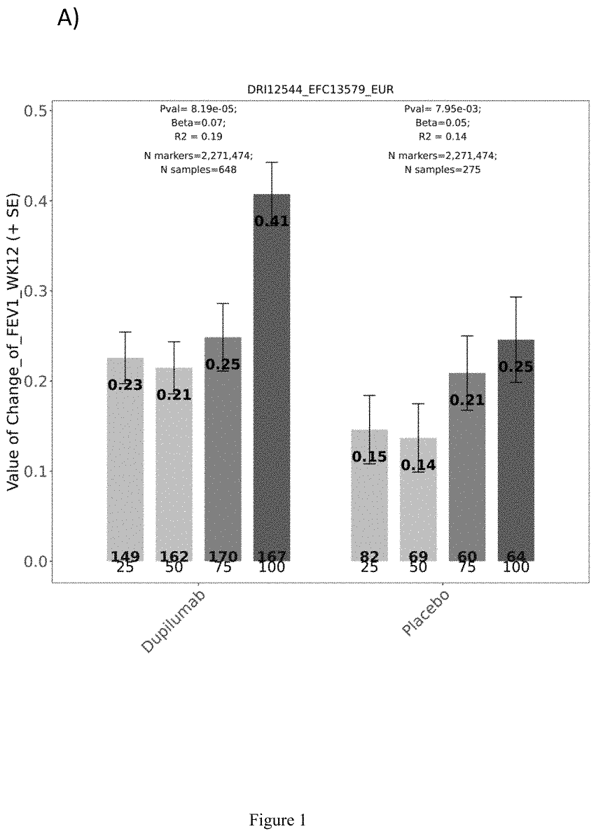

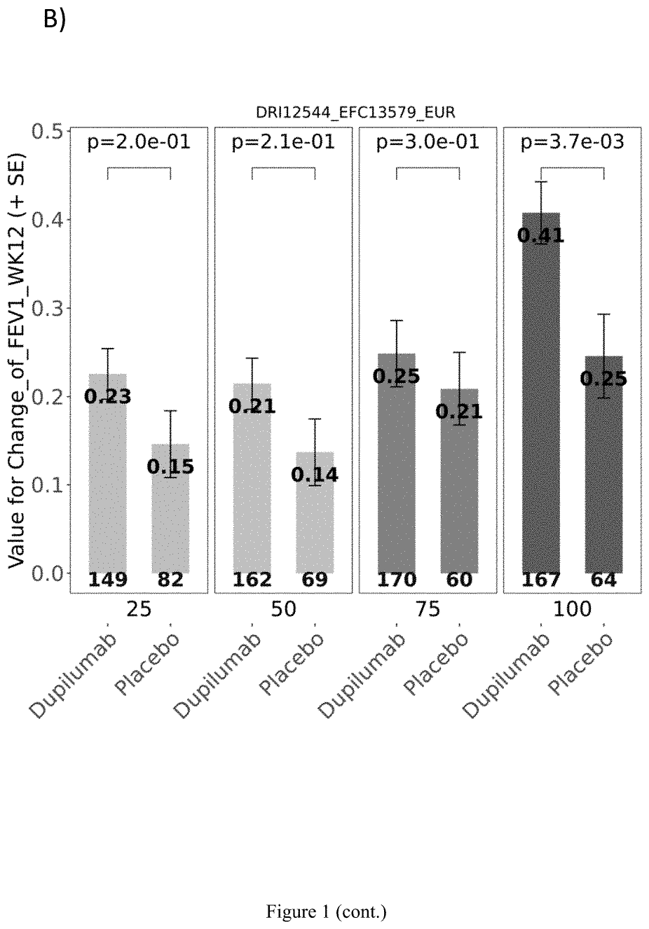

The graphs set forth in FIG. 1 (Panel A and Panel B) show that FEV1-PS was significantly associated with an increased change of FEV1 at week 12 in both dupilumab-treated and placebo-treated European subjects. The covariates were AGE, SEX, STUDYID (study identifier), ICS_FL (taking inhaled corticosteroids at baseline; yes/no), EOSBL_300 FL (eosinophils >300 at baseline), ASMANUM_LOG (exacerbation history for the previous year), HGTBL (height baseline), FEV1BL, and PC1-4 (principal components 1-4). The FEV1-PS was divided into quartiles and ranked from lowest to highest, the number at the bottom of each bar is the number of subjects in each group, and the number inside each bar is the change in FEV1 values at week 12. Applying the FEV1-PS to the population of dupilumab-treated subjects of European ancestry, it was observed that this FEV1-PS is significantly associated with better FEV1 improvement in both dupilumab and placebo arms.

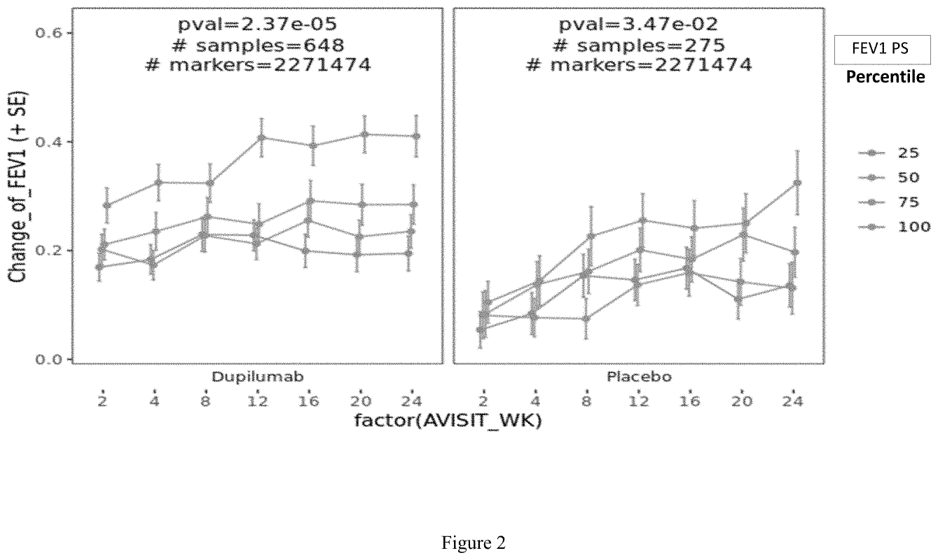

The graphs set forth in FIG. 2 show that FEV1-PS was significantly associated with a longitudinal increase of change of FEV1 in both dupilumab-treated and placebo European subjects. The covariates were AGE, SEX, STUDYID, ICS_FL, EOSBL_300 FL, ASMANUM_LOG, HGTBL, FEV1BL, and PC1-4. It was observed that the FEV1-PS is significantly associated with better FEV1 improvement in both dupilumab and placebo arms.

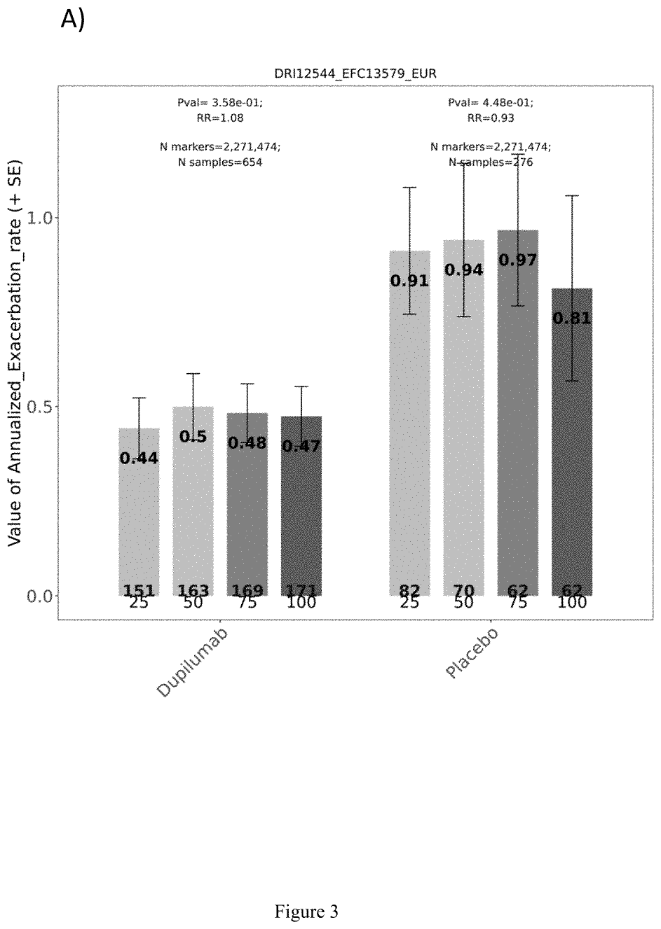

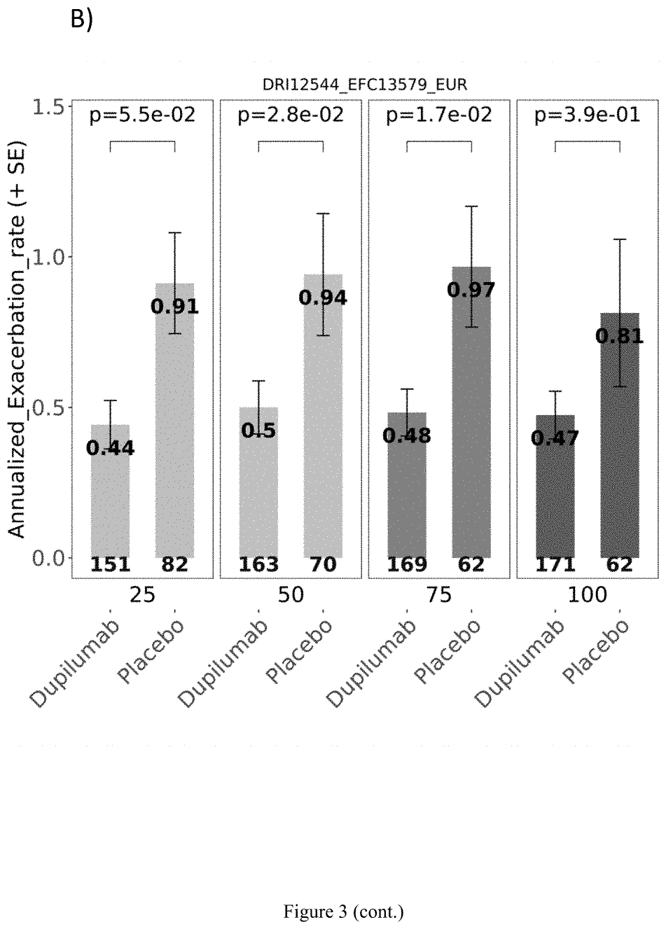

The graphs set forth in FIG. 3 (Panel A and Panel B) show that FEV1-PS was not associated with a higher annualized exacerbation rate in European subjects. The covariates were AGE, SEX, STUDYID, ICS_FL, EOSBL_300 FL, ASMANUM_LOG, HGTBL, FEV1BL, and PC1-4. The FEV1-PS was divided into quartiles and ranked from lowest to highest, the number at the bottom of each bar is the number of subjects in each group, and the number inside each bar is the change in annualized exacerbation rate.

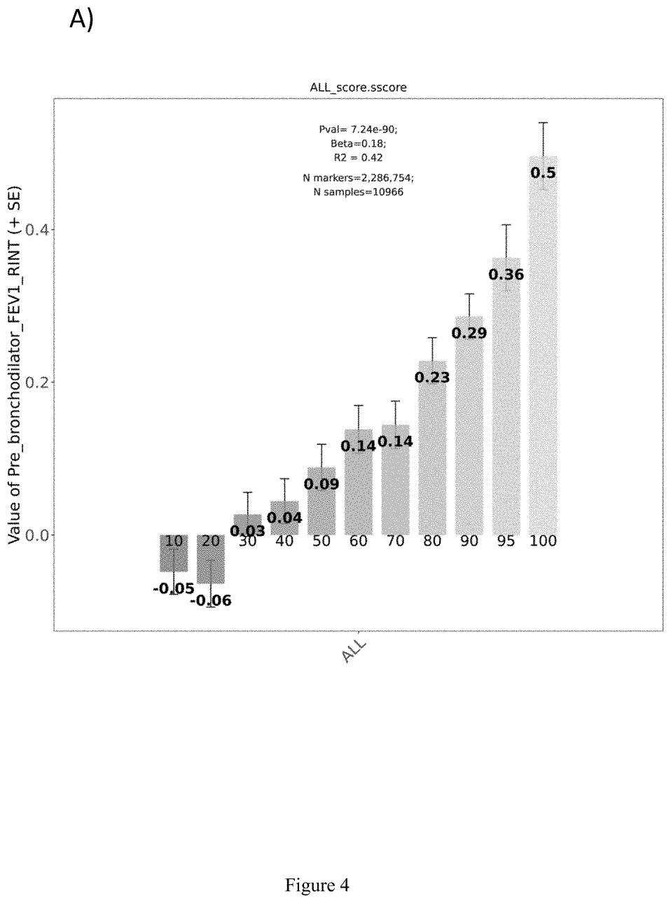

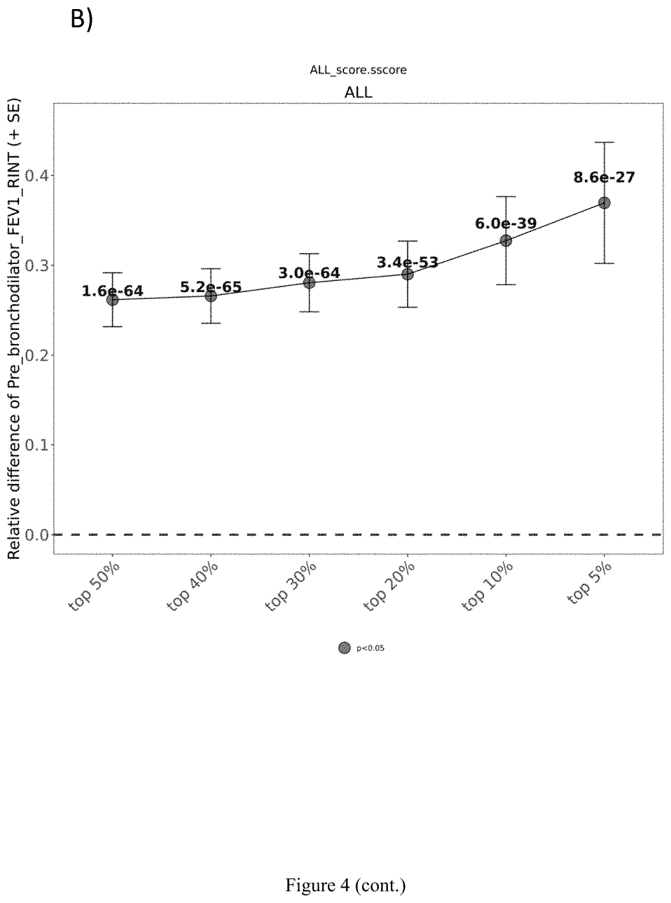

The graphs set forth in FIG. 4 (Panel A and Panel B) show that the FEV1-PS determined from European samples in the U.K. Biobank is significantly associated with pre-bronchodilator FEV1 in GHS_GSA European samples. For the graph in the first panel, the numbers “10,” “20,” “30,” “40,” “50,” “60,” “70,” “80,” “90,” “95,” and “100” are the FEV1-PS divided into deciles and ranked from lowest to highest and the values within the error bars are the values of pre-bronchodilator FEV1 response values that have undergone rank-based inverse normal transformation (RINT). For the graph in Panel B, the x-axis shows the cutoff for top percentiles of the FEV1-PS, and the y-axis shows the relative difference of pre-bronchodilator FEV1 RINT values, and the numbers within the error bars are the p-values comparing each respective top percentile cohort to each of the lower FEV1-PS percentile groups combined. The best performing method for FEV1-PS was found to be determined using the SBayesR method. This is another Bayesian method for estimating single-SNP contribution to traits and for developing PS.

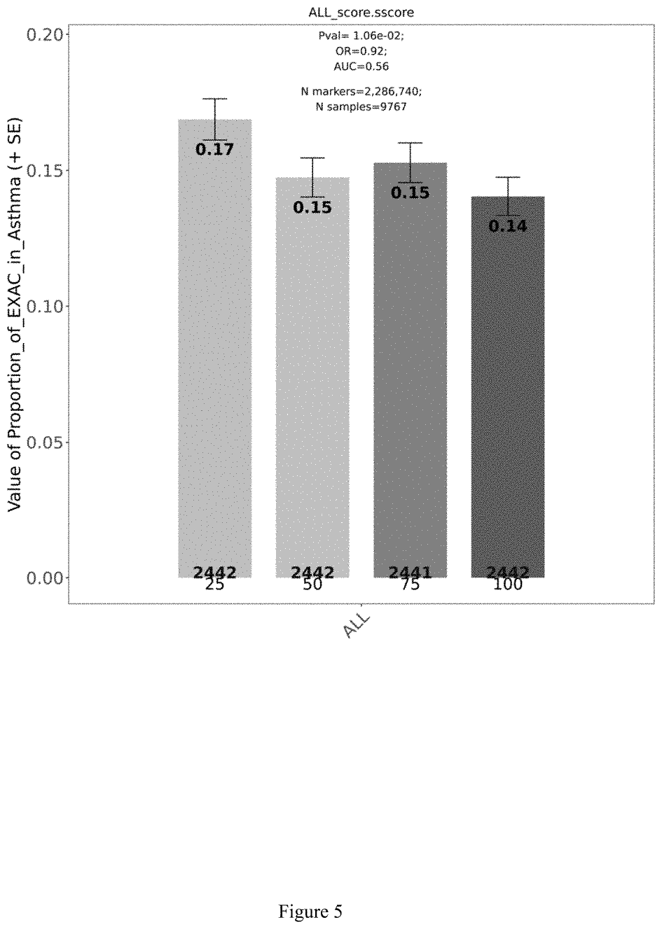

FIG. 5 shows a genome-wide FEV1-PS in (GHS_GSA European subjects) asthmatic patient and the percentage that have exacerbations. A p-value of 9.17e-28 was observed. The FEV1-PS divided into quartiles and ranked from lowest to highest and the number at the bottom of each bar is the number of subjects in each group.

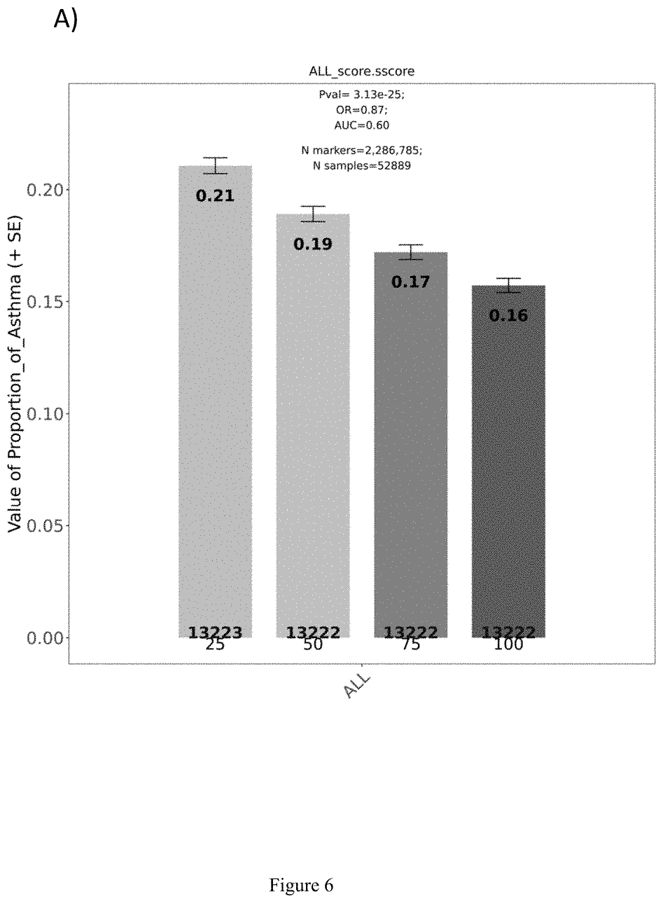

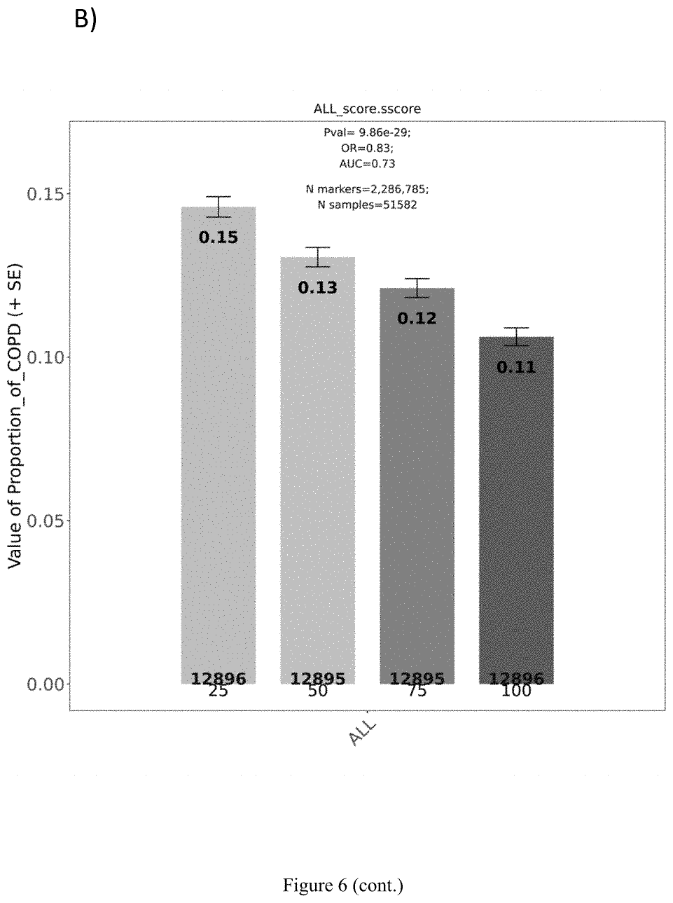

FIG. 6 shows a genome-wide FEV1-PS in GHS_GSA European subjects versus asthma and COPD. The values for asthma were associated with a p-value of 3.13e-25 and those for COPD were associated with a p-value of 9.86e-29. For the graph in Panel A, the FEV1-PS was divided into quartiles and ranked from lowest to highest, the number at the bottom of each bar is the number of subjects in each group, and the number at the top represents the percentage of patients with asthma from the total population. For the graph in Panel B, the FEV1-PS was divided into quartiles and ranked from lowest to highest, the number at the bottom of each bar is the number of subjects in each group, and the number at the top represents the percentage of patients with COPD from the total population.

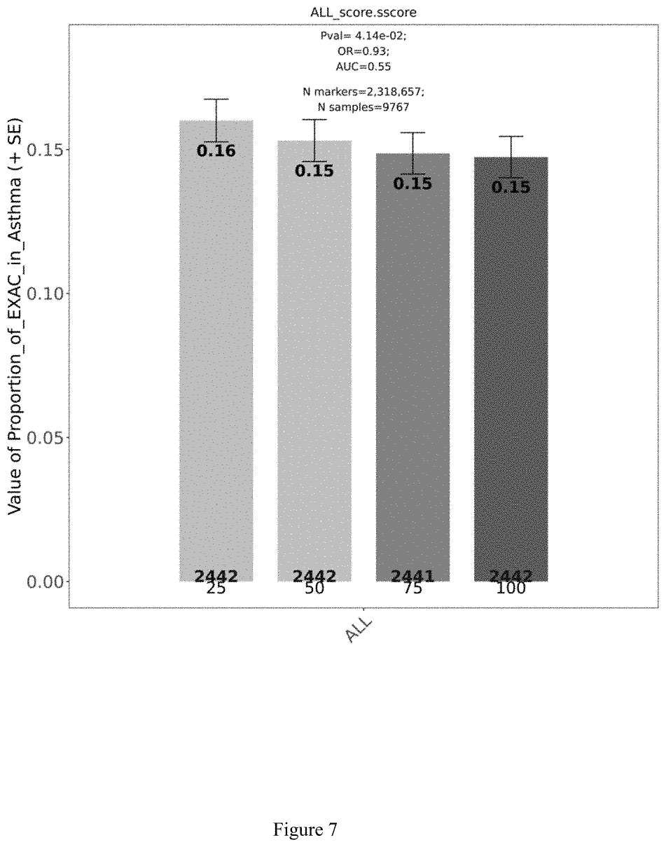

FIG. 7 shows a genome-wide FEV1-PS (a GWAS based on FEV1 values) developed in GHS_GSA European patients without asthma and COPD. The values for proportions of exacerbation in asthmatic patients were associated with a p-value of 4.14e-02. The FEV1-PS was divided into quartiles and ranked from lowest to highest, the number at the bottom of each bar is the number of subjects in each group, and the number at the top of each graph is the value of proportion of exacerbation in asthmatic patients.

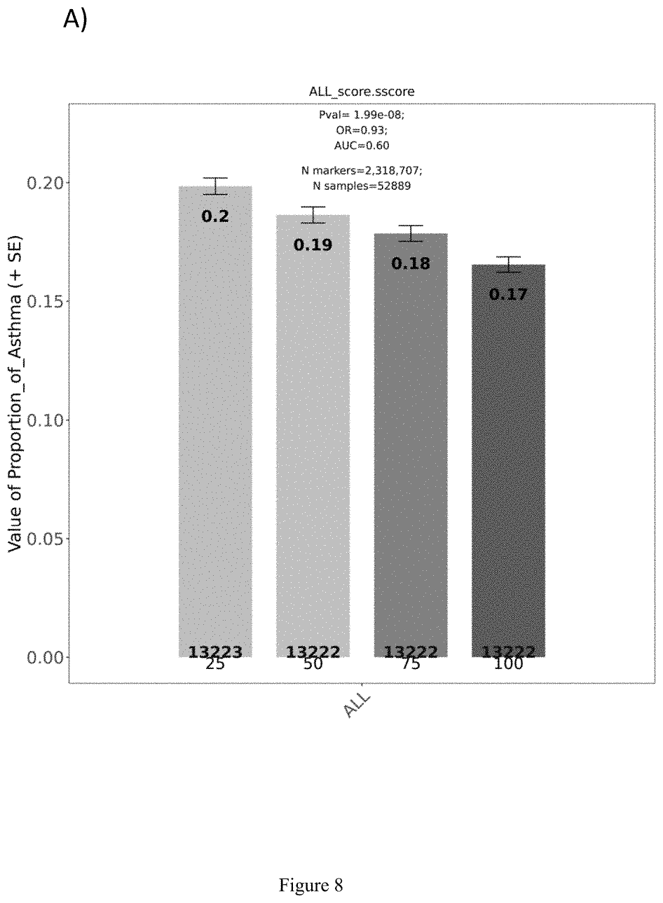

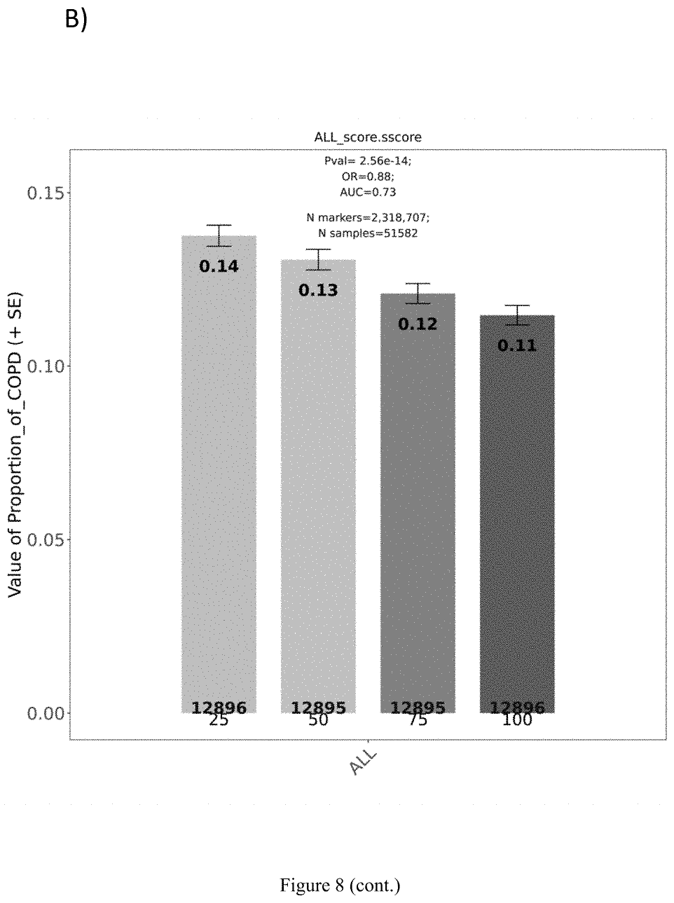

FIG. 8 (Panel A and Panel B) shows a genome-wide FEV1-PS (a GWAS based on FEV1 values) developed in GHS_GSA European patients without asthma and COPD. The values for proportion of asthmatic patients were associated with a p-value of 1.99e-08 and those for COPD were associated with a p-value of 2.56e-14. For the graph in Panel A, the FEV1-PS was divided into quartiles and ranked from lowest to highest, the number at the bottom of each bar is the number of subjects in each group, and the number at the top of each graph is the value of proportion of patients with asthma from the total population. For the graph in Panel B, the FEV1-PS was divided into quartiles and ranked from lowest to highest, the number at the bottom of each bar is the number of subjects in each group, and the number at the top of each graph is the value of proportion of patients with COPD from the total population.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

1. A method of treating a subject having a lung disease or at risk of developing a lung disease with a therapeutic agent that treats or inhibits a lung disease, the method comprising:
determining or having determined the subject's FEV1 polygenic score (FEV1-PS), wherein the FEV1-PS comprises an aggregate of a plurality of genetic variants associated with pre-bronchodilator FEV1; and
administering or continuing to administer the therapeutic agent that treats or inhibits a lung disease at a standard dosage amount when the subject's FEV1-PS is greater than or equal to a threshold FEV1-PS; or
administering the therapeutic agent that treats or inhibits a lung disease at dose that is greater than a standard dosage amount when the subject's FEV1-PS is less than the threshold FEV1-PS.

2. A method of determining whether a subject having a lung disease or at risk of developing a lung disease will adequately respond to treatment with a therapeutic agent that treats or inhibits a lung disease, the method comprising:
determining or having determined the subject's FEV1 polygenic score (FEV1-PS), wherein the FEV1-PS comprises an aggregate of a plurality of genetic variants associated with pre-bronchodilator FEV1; wherein:
an FEV1-PS that is greater than or equal to a threshold FEV1-PS indicates the subject will respond adequately to treatment with the therapeutic agent that treats or inhibits a lung disease at a standard dosage amount; or
an FEV1-PS that is less than a threshold FEV1-PS indicates the subject will not respond adequately to treatment with a standard dosage amount of the therapeutic agent that treats or inhibits a lung disease; and
administering an interleukin-4 receptor alpha antagonist and/or an interleukin-13 receptor antagonist other than dupilumab to a subject having an FEV1-PS that is less than the threshold FEV1-PS.

3. The method according to claim 1, wherein the lung disease is an obstructive lung disease, a restrictive lung disease, asthma, chronic obstructive pulmonary disease (COPD), and/or pulmonary fibrosis.

4-7. (canceled).

8. The method according to claim 1, wherein the therapeutic agent that treats or inhibits a lung disease is an interleukin-4 receptor alpha antagonist and/or an interleukin-13 receptor antagonist.

9. The method according to claim 8, wherein the interleukin-4 receptor alpha antagonist and/or an interleukin-13 receptor antagonist is dupilumab.

10. The method according to claim 1, wherein the therapeutic agent that treats or inhibits a lung disease is an IL-33 antagonist.

11. The method according to claim 10, wherein the IL-33 antagonist is itepekimab.

12. The method according to claim 1, wherein the therapeutic agent that treats or inhibits a lung disease is a bronchodilator.

13. The method according to claim 12, wherein the bronchodilator is an inhaled corticosteroid (ICS) or a long-acting beta agonist (LABA).

14. (canceled).

15. The method according to claim 1, wherein the method comprises administering an interleukin-4 receptor alpha antagonist and/or an interleukin-13 receptor antagonist to a subject having an FEV1-PS that is less than the threshold FEV1-PS.

16. The method according to claim 15, wherein the interleukin-4 receptor alpha antagonist and/or an interleukin-13 receptor antagonist is dupilumab.

17. The method according to claim 1, wherein the method comprises administering an interleukin-4 receptor alpha antagonist and/or an interleukin-13 receptor antagonist other than dupilumab to a subject having an FEV1-PS that is less than the threshold FEV1-PS.

18. The method according to claim 1, wherein the method comprises administering an IL-33 antagonist to a subject having an FEV1-PS that is less than the threshold FEV1-PS.

19. The method according to claim 18, wherein the IL-33 antagonist is itepekimab.

20-21. (canceled).

22. The method according to claim 1, wherein the method further comprises administering an IL-33 antagonist to a subject having an FEV1-PS that is less than the threshold FEV1-PS.

23. The method according to claim 22, wherein the IL-33 antagonist is itepekimab.

24. The method according to claim 1, wherein the threshold FEV1-PS is the top quartile within a reference population, the top quintile within a reference population, or the top decile within a reference population.

25-26. (canceled).

27. The method according to claim 24, wherein the reference population comprises at least 100 subjects.

28-30. (canceled).

31. The method according to claim 24, wherein the reference population is enriched for members of an ancestry group.

32. The method according to claim 31, wherein the ancestry group comprises a European ancestry group, an African ancestry group, an admixed American ancestry group, an East Asian ancestry group, or a South Asian ancestry group.

33-34. (canceled).

35. The method according to claim 1, wherein the plurality of genetic variants comprises a single nucleotide polymorphism, an insertion, a deletion, a structural variant, or a copy-number variation, or any combination thereof.

36. The method according to claim 1, wherein the plurality of genetic variants is determined by calculating a genetic variant performance in the reference population and selecting the highest performing genetic variants.

37. (canceled).

38. The method according to claim 1, wherein the FEV1-PS is calculated using an LDPred or an SBayesR method.

39. The method according to claim 1, wherein the FEV1-PS is calculated using a pruning and thresholding method.

40. The method according to claim 1, wherein the plurality of genetic variants comprises at least 2 genetic variants, at least 10 genetic variants, at least 25 genetic variants, at least 50 genetic variants, at least 100 genetic variants, at least 150 uenctic variants, at least 1,000 ucnetic variants, at least 10,000 ucnetic variants, at least 100,000 genetic variants, at least 1,000,000 genetic variants, at least 2,000,000 genetic variants, at least 5,000,000 genetic variants, or at least 10,000,000 genetic variants.

41-52. (canceled).

53. The method according to claim 1, wherein the FEV1-PS is determined from a biological sample obtained from the subject, wherein the biological sample comprises blood, semen, saliva, urine, feces, hair, teeth, bone, tissue, a swab from a cheek, or a cell.

54. (canceled).


SEQUENCE LISTING (10 sequences)

SEQ ID NO: 1 - protein, 124 aa, synthetic construct
  REGION 1..124: note = Dupilumab HCVR
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 2 - protein, 112 aa, synthetic construct [sequence omitted]

SEQ ID NO: 3 - protein, 8 aa, synthetic construct
  REGION 1..8: note = Dupilumab HCDR1
GFTFRDYA

SEQ ID NO: 4 - protein, 8 aa, synthetic construct
  REGION 1..8: note = Dupilumab HCDR2
ISGSGGNT

SEQ ID NO: 5 - protein, 18 aa, synthetic construct
  REGION 1..18: note = Dupilumab HCDR3
AKDRLSITIRPRYYGLDV

SEQ ID NO: 6 - protein, 11 aa, synthetic construct
  REGION 1..11: note = Dupilumab LCDR1
QSLLYSIGYNY

SEQ ID NO: 7 - skipped

SEQ ID NO: 8 - protein, 9 aa, synthetic construct
  REGION 1..9: note = Dupilumab LCDR3
MQALQTPYT

SEQ ID NO: 9 - protein, 451 aa, synthetic construct
  REGION 1..451: note = Dupilumab heavy chain
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 219 aa, synthetic construct
  REGION 1..219: note = Dupilumab light chain
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC